(19) United States
(12) Patent Application Publication
(10) Pub. No.: US 2008/0221088 A1
(43) Pub. Date: Sep. 11, 2008

(54) 3,4-Substituted Thiazoles as Ampk Activators

(76) Inventors: Vijay Kumar Potluri, Hyderabad (IN); Saibal Kumar Das, Hyderabad (IN); Pradip Kumar Sasmal, Hyderabad (IN); Javed Iqbal, Hyderabad (IN); Parimal Misra, Hyderabad (IN); Ranjan Chakrabarti, Hyderabad (IN); Rashmi Talwar, Hyderabad (IN)

Correspondence Address: Reddy Us Therapeutics, Inc, 3065 Northwoods Circle, Norcross, GA, 30071, US

(21) Appl. No.: 11/917,229

(22) PCT Filed: Jun. 23, 2006
(86) PCT No.: PCT/US06/24572

§ 371 Date: Dec. 12, 2007

Related U.S. Application Data
(60) Provisional application No. 60/752,683, filed on Dec. 21, 2005.

(30) Foreign Application Priority Data
Jun. 23, 2005 (IN) 788/CHE/2005

(51) Int. Cl. A61K 31/541; C07D 277/587; C07D 401/12; C07D 417/12; A61K 31/427; A61K 31/4439

(52) U.S. Cl. 514/227.8; 548/194; 546/274.7; 544/60; 514/370; 514/342

(57) ABSTRACT

The present application provides novel thiazole derivatives that are useful as activators of Adenosine 5′-Monophosphate-Activated Protein Kinase and pharmaceutical compositions containing such compounds.

### FIELD

The present application provides novel thiazole derivatives that are Adenosine 5′-Monophosphate-Activated Protein Kinase activators and pharmaceutical compositions containing such compounds.

### BACKGROUND

Adenosine 5′-Monophosphate-Activated Protein Kinase (AMP activated protein kinase) or (AMPK) activators are believed to play a key role in regulation of carbohydrates and fat metabolism in mammals including humans. The net effects of AMPK activation may include inhibition of hepatic gluconeogenesis, cholesterol and triglyceride synthesis in liver, enhancement in muscle glucose transport and insulin sensitivity and fatty acid oxidation in muscle and liver.

### SUMMARY

In accordance with one aspect, the present application provides thiazole derivatives having the general formula (I), which are a free species and/or a pharmaceutically-acceptable salt or a solvate or a hydrate thereof:

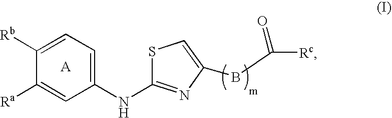

where R^{a} and R^{b}, which may be same or different, are independently chosen from fluoro, chloro, bromo, nitro, (C₁-C₅) perfluoroalkyl, (C₁-C₅) alkyl, (C₁-C₅) alkoxy, (C₁-C₅) perfluoroalkoxy, benzyl, cyano and hydroxy;

B is —CH₂, —CH(CH₃)— or —C(CH₃)₂—;

m varies between 0 and 2, inclusive;

with a proviso that R^{a} and R^{b} are not both chloro; and

R^{c} is —OR₁ or —NR₂R₃, wherein R₁ is hydrogen or (C₁-C₈) alkyl, and R₂ and R₃ are designated according to alternatives a) or b). In the alternative a), R₂ is hydrogen and R₃ is chosen from hydrogen, NH₂, (C₁-C₅) alkyl, (C₁-C₅) alkylaryl, aryl, heteroaryl, and (C₁-C₅) alkylheteroaryl. In the alternative b), R₂ and R₃, together with the nitrogen atom of the group —NR₂R₃, form a 5- or 6-membered heterocycloalkyl group. Various embodiments and variants are provided.

In accordance with another aspect, the present application also provides thiazole derivatives of the general formula (II), which are a free species and/or a pharmaceutically-acceptable salt thereof:

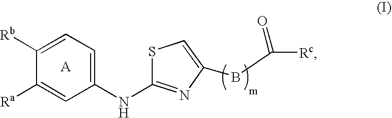

where R^{a} and R^{b}, which may be same or different, are independently chosen from fluoro, chloro, bromo, nitro, (C₁-C₅) perfluoroalkyl, (C₁-C₅) alkyl, (C₁-C₅) alkoxy, (C₁-C₅) perfluoroalkoxy, benzyl, cyano and hydroxy;

B is —CH₂, —CH(CH₃)— or —C(CH₃)₂—;

m varies between 0 and 2, inclusive;

with a proviso that R^{a} and R^{b} are not both chloro; and

R^{c} is —OR₁ or —NR₂R₃, wherein R₁ is hydrogen or (C₁-C₈) alkyl, and R₂ and R₃ are designated according to the alternatives a) or b); which thiazole derivative has AMP-activated protein kinase (AMPK) potential of at least about 80% in L6 muscle cells and of at least about 90% in Hep G2 muscle cells. Various embodiments and variants are provided.

In accordance with other aspects, the present application also provides a method of activating AMP-activated protein kinase (AMPK) in human or animal subject, said method comprising administering a subject with an effective amount of the thiazole derivative of the present application.

In yet another aspect, the present application provides pharmaceutical compositions comprising one or more thiazole derivatives of the present application and one or more pharmaceutically-acceptable excipients.

### DETAILED DESCRIPTION

To describe the present application, certain terms are defined herein as follows.

The term “compound” is used to denote a molecule of unique, identifiable chemical structure. A compound may exist as a free species. Also, the free species form of the compound may form various salts, usually with external acids or bases.

The term “derivative” is used as a common term for the free species form of the compound and all its salts. Thus, the claim language “a derivative, which is a free species and/or a salt of the compound of the formula [I]” is used to define a genus comprising the free species compounds of the given formula and all the salts of the compounds of the given formula. The use of the term “and/or” is intended to indicate that, for a compound of a given chemical structure, a claim to a “derivative” covers the free species form and all of its salts, as well as the mixtures of free species and the salt forms. The term “pharmaceutically-acceptable salts” is intended to denote salts that are suitable for use in human or animal pharmaceutical products. The use of the term “pharmaceutically-acceptable” is not intended to limit the claims to substances (“derivatives”) found only outside of the body.

In describing the compounds, certain nomenclature and terminology is used throughout to refer to various groups and substituents. The description “Cₓ-C_{y}” refers to a chain of carbon atoms or a carbocyclic skeleton containing from x to y atoms, inclusive. The designated range of carbon atoms may refer independently to the number of carbon atoms in the chain or the cyclic skeleton, or to the portion of a larger substituent in which the chain or the skeleton is included. For example, the recitation “(C₁-C₅) alkyl” refers to an alkyl group having a carbon chain of 1 to 5 carbon atoms, inclusive of 1 and 5. The chains of carbon atoms of the groups and substituents described and claimed herein may be saturated or unsaturated, straight chain or branched, substituted or unsubstituted.

The term “alkyl,” whether used alone or as a part of another group, refers to a group or a substituent that includes a chain of carbon atoms. The chains of carbon atoms of the alkyl groups described and claimed herein may be saturated or unsaturated, straight chain or branched, substituted or unsubstituted. In a non-limiting example, “C₁-C₅ alkyl” denotes an alkyl group having carbon chain with from 1 to 5 carbon atoms, inclusive, which carbon may be saturated or unsaturated, straight chain or branched, substituted or unsubstituted. The term “perfluoroalkyl” is used to denote an alkyl group in which all hydrogen atoms had been replaced with fluorine atoms, for example trifluoromethyl group.

The term “alkoxy” refers to an oxygen ether radical. An “alkoxy” group contains a chain of carbon atoms connected to the rest of the molecule through the oxygen atom. The chains of carbon atoms of the alkoxy groups described and claimed herein may be saturated or unsaturated, straight chain or branched, substituted or unsubstituted. The term “perfluoroalkoxy” is used to denote an alkoxy group in which all hydrogen atoms had been replaced with fluorine atoms, for example trifluoromethoxy group.

The term “aryl”, whether used alone or as part of a substituent group, denotes a carbocyclic aromatic radical derived from an aromatic hydrocarbon. Non-limiting examples of the “aryl” radicals include phenyl, naphthyl, diphenyl, fluorophenyl, methoxyethylphenyl, difluorophenyl, benzyl, benzoyloxyphenyl, carboethoxyphenyl, acetylphenyl, ethoxyphenyl, phenoxyphenyl, hydroxyphenyl, carboxyphenyl, trifluoromethylphenyl, tolyl, xylyl, and dimethylcarbamylphenyl. The “aryl” groups of the compounds described herein may be substituted by independent replacement of 1 to 3 of the hydrogen atoms on the carbocyclic aromatic skeleton with substituents including, but not limited to, halogen, —OH, —CN, mercapto, nitro, amino, substituted amino, C₁-C₆ alkyl, C₁-C₆ alkoxy, C₁-C₆ alkylthio, C₁-C₆ alkylamino, halogenated C₁-C₆ alkyl, formyl, C₁-C₆ acyl, C₁-C₆ alkoxyacyl, and C₁-C₆ acylamido.

The term “alkylaryl” is used to denote a group comprised of an aryl radical and a carbon chain that connects the aryl radical to the rest of the molecule, for example benzyl group.

The term “heteroaryl”, whether used alone or as part of a substituent group, is used to denote a cyclic aromatic radical having from five to ten ring atoms of which at least one ring atom is heteroatom, i.e., it is not a carbon atom. For example, there are from 1 to 4 heteroatoms in the ring structure selected from S, O, and N. The radical may be joined to the rest of the molecule via any of the ring atoms. Non-limiting examples of “heteroaryl” groups include pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl. The heteroaryl groups of the compounds described and/or claimed herein may be substituted by independent replacement of 1 to 3 hydrogen atoms of the aromatic skeleton with substituents including, but not limited to halogen, —OH, —CN, mercapto, nitro, amino, substituted amino, C₁-C₆ alkyl, C₁-C₆ alkoxy, C₁-C₆ alkylthio, C₁-C₆ alkylamino, halogenated C₁-C₆ alkyl, formyl, C₁-C₆ acyl, C₁-C₆ alkoxyacyl, and C₁-C₆ acylamido.

The term “alkylheteroaryl” is used to denote a group comprised of a heteroaryl radical and a carbon chain that connects the heteroaryl radical to the rest of the molecule, for example methylpyridyl group.

The term “heterocycloalkyl”, whether used alone or as part of a substituent group, is used to denote a cyclic non-aromatic radical having from five to ten ring atoms of which at least one ring atom is heteroatom, i.e., it is not a carbon atom. For example, there are from 1 to 4 heteroatoms in the ring structure selected from S, O, and N. Non-limiting examples of heterocycloalkyl are aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4-dioxaspiro[4.3]octyl, and 1,4-dioxaspiro[4.2]heptyl 5- or 6-membered heterocycloalkyl group formed by R₂ and R₃ are morpholine, thiomorpholine and the like.

Unless specified otherwise, it is intended that the definition of any substituent or variable at a particular location in a molecule be independent of its definitions elsewhere in that molecule. It is understood that substituents and substitution patterns on the compounds of this application can be selected by one of ordinary skill in the art to provide compounds that are chemically stable and that can be readily synthesized by techniques known in the art as well as those methods set forth herein.

A group may be referred to generally or more specifically, as desired. For example, a group containing a carbon chain with one carbon-carbon double bond may be described as alkyl or alkenyl, as desired. In another non-limiting example, a group containing a carbon chain with a chloro substituent may be described as alkyl or halogenated alkyl, as desired.

A “composition” may contain one compound or a mixture of compounds. A “pharmaceutical composition” is any composition useful or potentially useful in producing physiological response in a subject to which such pharmaceutical composition is administered. The term “pharmaceutically acceptable” with respect to excipients is used to define non-toxic substances generally suitable for use in human or animal pharmaceutical products.

AMPK activation potential percentages are obtained by normalizing the values obtained at the concentrations in the range of 2 nM-10 μM with that of metformin by considering the values obtained for metformin at 2 mM concentrations as 100%.

One embodiment of the present application provides compounds of formula (I)

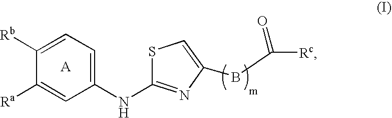

where R^{a} and R^{b}, which may be same or different, are independently chosen from fluoro, chloro, bromo, nitro, (C₁-C₅) perfluoroalkyl, (C₁-C₅) alkyl, (C₁-C₅) alkoxy, (C₁-C₅) perfluoroalkoxy, benzyl, cyano and hydroxy;

B is —CH₂, —CH(CH₃)— or —C(CH₃)₂—;

m varies between 0 R^{a} and R^{b} are not both chloro; and

R³ is —OR₁ or —NR₂R₃, wherein R₁ is hydrogen or (C₁-C₈) alkyl, and R₂ and R₃ are designated according to alternatives a) or b). In the alternative a), R₂ is hydrogen and R₃ is chosen from hydrogen, NH₂, (C₁-C₅) alkyl, (C₁-C₅) alkylaryl, aryl, heteroaryl, and (C₁-C₅) alkylheteroaryl. In the alternative b), R₂ and R₃, together with the nitrogen atom of the group —NR₂R₃, form a 5- or 6-membered heterocycloalkyl group.

Another embodiment of the present application provides compounds of formula (II)

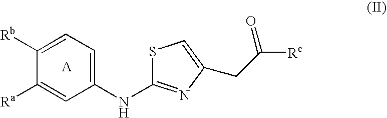

where R^{a} and R^{b}, which may be same or different, are independently chosen from fluoro, chloro, bromo, nitro, (C₁-C₅) perfluoroalkyl, (C₁-C₅) alkyl, (C₁-C₅) alkoxy, (C₁-C₅) perfluoroalkoxy, benzyl, cyano and hydroxy;

m varies between 0 and 2, inclusive;

with a proviso that R^{a} and R^{b} are not both chloro; and

R³ is —OR₁ or —NR₂R₃, wherein R₁ is hydrogen or (C₁-C₈) alkyl, and R₂ and R₃ are designated according to alternatives a) or b). In the alternative a), R₂ is hydrogen and R₃ is chosen from hydrogen, NH₂, (C₁-C₅) alkyl, (C₁-C₅) alkylaryl, aryl, heteroaryl, and (C₁-C₅) alkylheteroaryl. In the alternative b), R₂ and R₃, together with the nitrogen atom of the group —NR₂R₃, form a 5- or 6-membered heterocycloalkyl group.

Another embodiment of the present application provides compounds of formula (II), wherein R^{a} and R^{b} are independently chosen from (C₁-C₈)perfluoroalkyl, chloro, bromo, fluoro or methyl.

Another embodiment of the present application provides compounds of formula (II), wherein R^{a} is trifluoromethyl, R^{b} is (C₁-C₈)perfluoroalkyl, chloro, bromo, fluoro or methyl.

Another embodiment of the present application provides compounds of formula (II), wherein R^{a} is trifluormethyl, R^{b} is chloro or bromo.

Another embodiment of the present application provides compounds of formula (III),

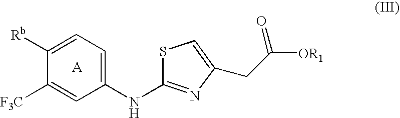

wherein R^{b} is fluoro or chloro, R₁ is hydrogen or (C₁-C₈)alkyl.

Another embodiment of the present application provides compounds of formula (III), wherein R₁ is hydrogen.

Another embodiment of the present application provides compounds of formula (IV)

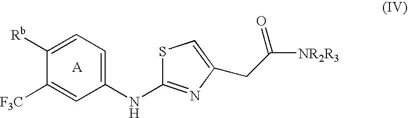

wherein R^{b} is fluoro or chloro;
R₂ is hydrogen;
R₃ is chosen from hydrogen, (C₁-C₅)alkyl, NH₂ or a group of the structure

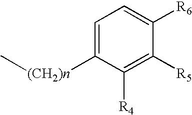

wherein R₄, R₅ and R₆ are independently chosen from chloro, bromo, fluoro, (C₁-C₅)alkyl, (C₁-C₅)alkoxy, (C₁-C₅)perfluoroalkyl, (C₁-C₅)alkylcarboxy, (C₁-C₅)alkylthio or (C₁-C₅)alkylacetomido and at least one of R₄, R₅ and R₆ is hydrogen; n varies from 0 to 3, inclusive; or
R₂ and R₃ together may form a 5 or 6-membered heterocyclic group along with the nitrogen atom to which they are attached and said ring may optionally contain 1 or 2 heteroatoms selected from oxygen, sulfur or nitrogen.

Another embodiment of the present application provides compounds of formula (IV), wherein R₂ is hydrogen and R₃ is (C₁-C₈)alkyl.

Another embodiment of the present application provides an ester prodrug of the thiazole derivatives of formula (I), wherein R₁ is not (C₁-C₈)alkyl.

Another embodiment of the present application provides an ester prodrug of the thiazole derivatives of formula (III), wherein R₁ is not (C₁-C₅)alkyl.

Another embodiment of the present application provides the thiazole derivatives of formula (I), which has the structure

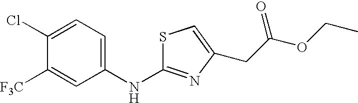

Another embodiment of the present application provides the thiazole derivatives of formula (I), which has the structure

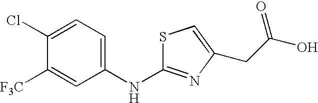

Another embodiment of the present application provides the thiazole derivatives of formula (I), which has the structure

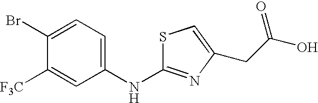

Another embodiment of the present application provides the thiazole derivatives of formula (I), which has the structure

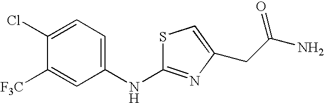

Another embodiment of the present application provides the thiazole derivatives of formula (I), which has the structure

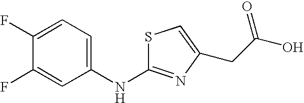

Another embodiment of the present application provides the thiazole derivatives of formula (I), which has the structure

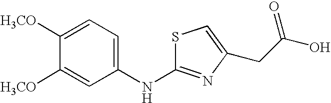

Another embodiment of the present application provides the thiazole derivatives of formula (I), which has the structure

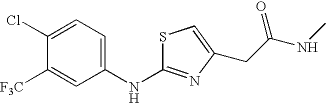

Another embodiment of the present application provides the thiazole derivatives of formula (I), which has the structure

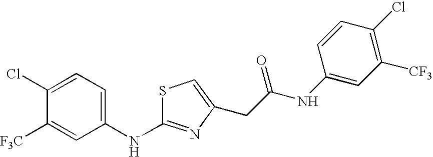

Another embodiment of the present application provides the thiazole derivatives of formula (I), which has the structure

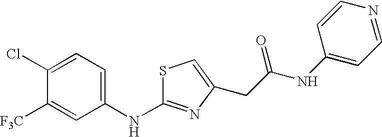

Another embodiment of the present application provides the thiazole derivatives of formula (I), which has the structure

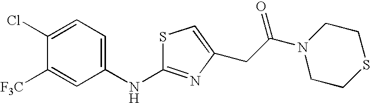

According to another embodiment of the present application provides, thiazole derivatives, which are free species and/or a pharmaceutically-acceptable salt of the compound of the formula (I).

According to another embodiment of the present application provides, novel thiazole derivatives of formula (I) which have a AMP kinase activation potential of at least about 80% in L6 muscle cells and of at least about 90% in Hep G2 muscle cells.

Another embodiment of the present application provides a method of activating AMPK in human or animal subject, said method comprising administering said subject with an effective amount of the thiazole derivative of compound of formula (I).

Another embodiment of the present application provides a pharmaceutical composition comprising a therapeutically effective amount of a compound of formula (I) in combination with a pharmaceutically suitable carrier.

Another embodiment of the present application provides a pharmaceutical composition comprising one or more thiazole derivatives of compound of formula (I) and one or more pharmaceutically-acceptable excipients.

Specific compounds of formula (I) include, but are not limited to:

| Structure | IUPAC Name |
|---|---|
| 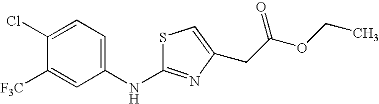 | [2-(4-Chloro-3-trifluoromethyl-phenylamino)-thiazol-4-yl]-acetic acid ethyl ester |
| 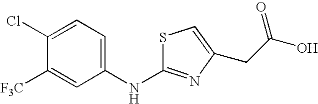 | [2-(4-Chloro-3-trifluoromethyl-phenylamino)-thiazol-4-yl]-acetic acid |
| 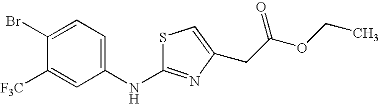 | [2-(4-Bromo-3-trifluoromethyl-phenylamino)-thiazol-4-yl]-acetic acid ethyl ester |
| 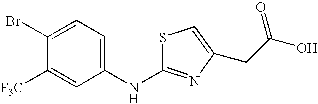 | [2-(4-Bromo-3-trifluoromethyl-phenylamino)-thiazol-4-yl]-acetic acid |
| 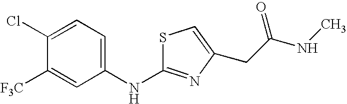 | 2-[2-(4-Chloro-3-trifluoromethyl-phenylamino)-thiazol-4-yl]-N-methyl-acetamide |
| 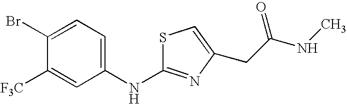 | 2-[2-(4-Bromo-3-trifluoromethyl-phenylamino)-thiazol-4-yl]-N-methyl-acetamide |
| 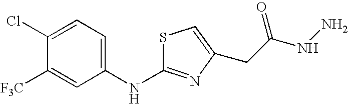 | [2-(4-Chloro-3-trifluoromethyl-phenylamino)-thiazol-4-yl]-acetic acid hydrazide |
| 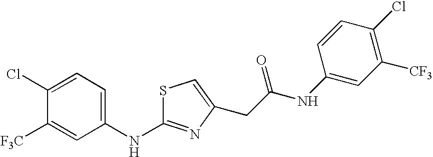 | N-(4-Chloro-3-trifluoromethyl-phenyl)-2-[2-(4-chloro-3-trifluoromethyl-phenylamino)-thiazol-4-yl]-acetamide |
| 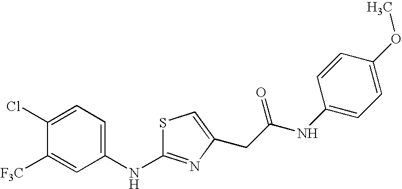 | 2-[2-(4-Chloro-3-trifluoromethyl-phenylamino)-thiazol-4-yl]-N-(4-methoxy-phenyl)-acetamide |
| 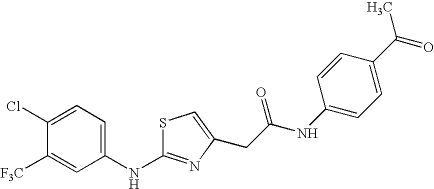 | N-(4-Acetyl-phenyl)-2-[2-(4-chloro-3-trifluoromethyl-phenylamino)-thiazol-4-yl]-acetamide |
| 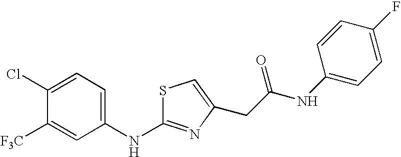 | 2-[2-(4-Chloro-3-trifluoromethyl-phenylamino)-thiazol-4-yl]-N-(4-fluoro-phenyl)-acetamide |
| 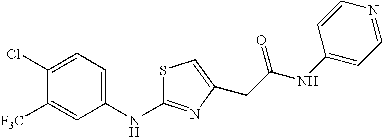 | 2-[2-(4-Chloro-3-trifluoromethyl-phenylamino)-thiazol-4-yl]-N-pyridin-4-yl-acetamide |
| 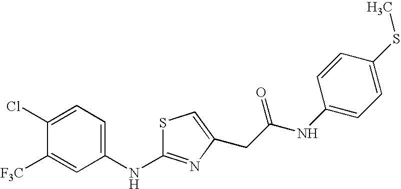 | 2-[2-(4-Chloro-3-trifluoromethyl-phenylamino)-thiazol-4-yl]-N-(4-methylsulfanyl-phenyl)-acetamide |
| 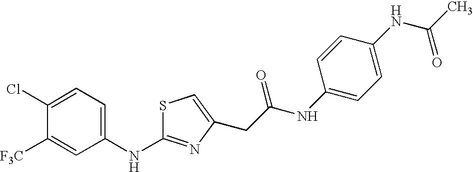 | N-(4-Acetylamino-phenyl)-2-[2-(4-chloro-3-trifluoromethyl-phenylamino)-thiazol-4-yl]-acetamide |
| 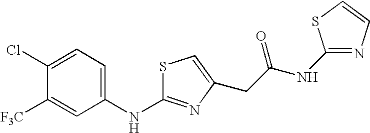 | 2-[2-(4-Chloro-3-trifluoromethyl-phenylamino)-thiazol-4-yl]-N-thiazol-2-yl-acetamide |
| 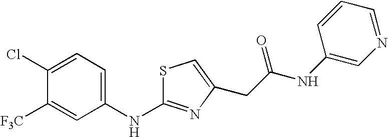 | 2-[2-(4-Chloro-3-trifluoromethyl-phenylamino)-thiazol-4-yl]-N-pyridin-3-yl-acetamide |
| 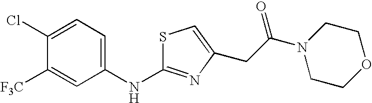 | 2-[2-(4-Chloro-3-trifluoromethyl-phenylamino)-thiazol-4-yl]-1-morpholin-4-yl-ethanone |
| 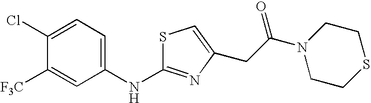 | 2-[2-(4-Chloro-3-trifluoromethyl-phenylamino)-thiazol-4-yl]-1-thiomorpholin-4-yl-ethanone |
| 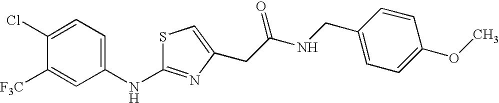 | 2-[2-(4-Chloro-3-trifluoromethyl-phenylamino)-thiazol-4-yl]-N-(4-methoxy-benzyl)-acetamide |
| 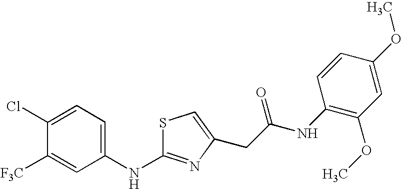 | 2-[2-(4-Chloro-3-trifluoromethyl-phenylamino)-thiazol-4-yl]-N-(2,4-dimethoxy-phenyl)-acetamide |
| 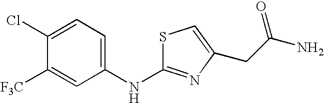 | 2-[2-(4-Chloro-3-trifluoromethyl-phenylamino)-thiazol-4-yl]-acetamide |
| 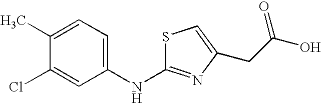 | [2-(3-Chloro-4-methyl-phenylamino)-thiazol-4-yl]-acetic acid |
| 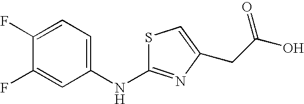 | [2-(3,4-Difluoro-phenylamino)-thiazol-4-yl]-acetic acid |
| 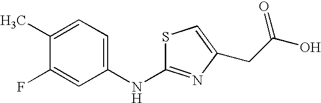 | [2-(3-Fluoro-4-methyl-phenylamino)-thiazol-4-yl]-acetic acid |
| 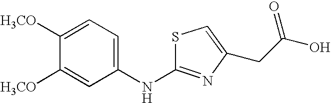 | [2-(3,4-Dimethoxy-phenylamino)-thiazol-4-yl]-acetic acid |
| 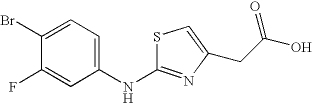 | [2-(4-Bromo-3-fluoro-phenylamino)-thiazol-4-yl]-acetic acid |
| 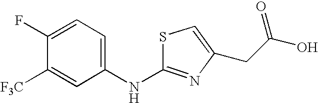 | [2-(4-Fluoro-3-trifluoromethyl-phenylamino)-thiazol-4-yl]-acetic acid |
| 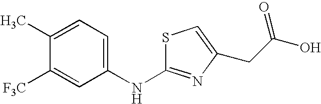 | [2-(4-Methyl-3-trifluoromethyl-phenylamino)-thiazol-4-yl]-acetic acid |
| 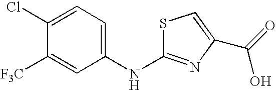 | 2-(4-Chloro-3-trifluoromethyl-phenylamino)-thiazole-4-carboxylic acid |
| 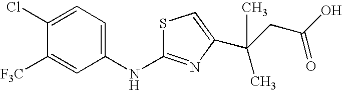 | 3-[2-(4-Chloro-3-trifluoromethyl-phenylamino)-thiazol-4-yl]-3-methyl-butyricacid |
| 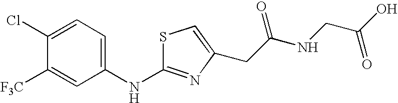 | {2-[2-(4-chloro-3-trifluoromethyl-phenylamino)-thiazol-4-yl]-acetylamino}-acetic acid |
| 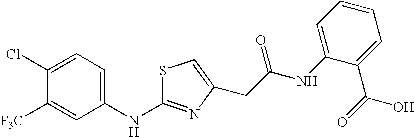 | 2-{2-[2-(4-Chloro-3-trifluoromethyl-phenylamino)-thiaol-4-yl}-acetylamino}-benzoic acid. |

Compounds of formula (I) can be prepared using known means, as for example, in the manner shown in the following preparation schemes.

The compounds of (I) which are thus prepared may be isolated and purified from the reaction mixture by known means, including solvent extraction, concentration, neutralization, filtration, crystallization, recrystallization, column chromatography, high performance liquid chromatography and recrystallization, to give a highly purified product of interest.

The compounds of the present application and salts thereof can be prepared by applying various synthetic methods utilizing the characteristics due to the fundamental skeleton or type of the substituents thereof. Representative production methods will be illustrated as hereunder. All other symbols are as defined earlier.

### Process 1

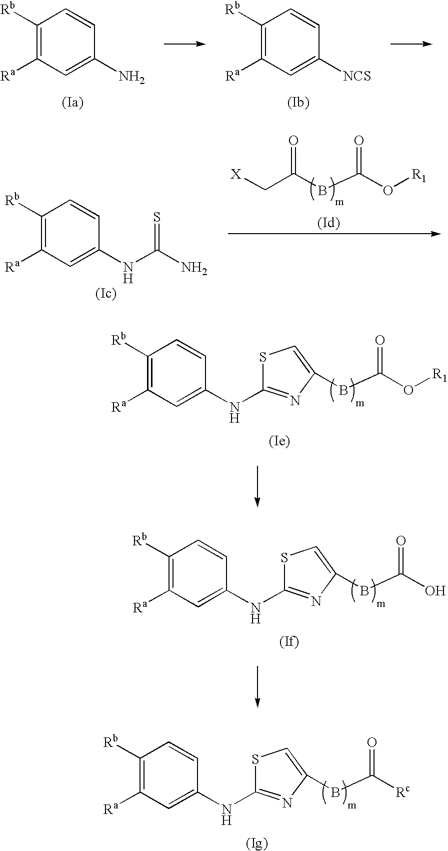

The compound of formula (Ia) was converted to a compound of formula (Ib) in presence of thiophosgene, pyridine and solvent, where all symbols are as defined earlier. The solvent used in the reaction can be selected from dichloromethane, dichloroethane, pyridine, chloroform, tetrachloromethane, ethylacetate, methanol, ethanol, isopropanol, n-propanol, butanol, acetone, acetonitrile, dimethylformamide, dimethylsulfoxide, tetrahydrofuran, water and the like or a mixture thereof. The reaction can be carried out at a temperature between about −10° C. to about 45° C. The duration of reaction can be maintained for a period in the range of about 5 minutes to about 3 hours, for example, about 30 minutes.

The compound of formula (Ib) was converted to a compound of formula (Ic) in presence of ammonia or aqueous ammonia solution and solvent, where all symbols are as defined earlier. The solvent used in the reaction can be selected from dichloromethane, dichloroethane, pyridine, chloroform, tetrachloromethane, ethylacetate, methanol, ethanol, isopropanol, n-propanol, butanol, acetone, acetonitrile, dimethylformamide, dimethylsulfoxide, tetrahydrofuran, water and the like or a mixture thereof. The reaction can be carried out at a temperature between about 10° C. to about 65° C. The duration of reaction can be maintained for a period in the range of about 1 hour to about 10 hours, for example, about 6 hours.

The compound of formula (Ic) was reacted with the compound of formula (Id) in presence of solvent; wherein X represents a leaving group such as halogen atom, p-toluenesulfonate, methanesulfonate, trifluoromethane sulfonate or the like and R₁ is selected from alkyl having 1 to 5 carbon atoms and may be, but is not limited to methyl, ethyl, n-propyl, iso-propyl or n-butyl, the other symbols are as defined earlier; to produce a compound of formula (Ie). The solvent used in the reaction can be selected from dichloromethane, dichloroethane, pyridine, chloroform, tetrachloromethane, ethylacetate, methanol, ethanol, isopropanol, n-propanol, butanol, acetone, acetonitrile, dimethylformamide, dimethylsulfoxide, tetrahydrofuran, water and the like or a mixture thereof. The reaction can be carried out at a temperature between about 40° C. to about 150° C. The duration of reaction can be maintained for a period in the range of about 6 hour to about 18 hours, for example, about 12 hours.

Hydrolysis of compound of formula (Ie) was converted to compound of formula (If), where all symbols are as defined earlier. The solvent used in the reaction can be selected from dichloromethane, dichloroethane, pyridine, chloroform, tetrachloromethane, ethylacetate, methanol, ethanol, isopropanol, n-propanol, butanol, acetone, acetonitrile, dimethylformamide, dimethylsulfoxide, tetrahydrofuran, water and the like or a mixture thereof. The hydrolysis can be carried out in presence of acid or base, for example in presence of base such as sodium hydroxide, potassium hydroxide, sodium carbonate, potassium carbonate and the like. The reaction can be carried out at a temperature between about 25° C. to about 75° C. The duration of reaction can be maintained for a period in the range of about 25 minutes to about 3 hours, for example, about 45 minutes.

The compound of formula (If) was converted to compound of formula (Ig) in presence of NH₂—R^{c}, where all symbols are as defined earlier. The solvent used in the reaction can be selected from dichloromethane, dichloroethane, pyridine, chloroform, tetrachloromethane, ethylacetate, methanol, ethanol, isopropanol, n-propanol, butanol, acetone, acetonitrile, dimethylformamide, dimethylsulfoxide, tetrahydrofuran, water and the like or a mixture thereof. The reaction can be carried out at a temperature between about 25° C. to about 75° C. The duration of reaction can be maintained for a period in the range of about 1 hour to about 5 hours, for example about 2 hours.

### Process 2

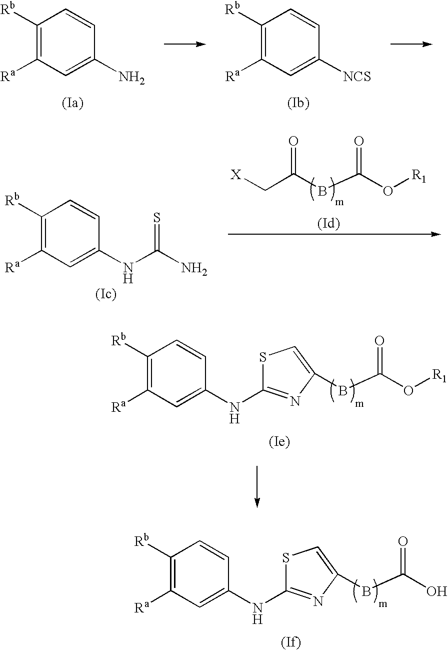

The compound of formula (Ia) was converted to a compound of formula (Ib) in presence of thiophosgene and pyridine, where all symbols are as defined earlier. The solvent used in the reaction can be selected from dichloromethane, dichloroethane, pyridine, chloroform, tetrachloromethane, ethylacetate, methanol, ethanol, isopropanol, n-propanol, butanol, acetone, acetonitrile, dimethylformamide, dimethylsulfoxide, tetrahydrofuran, water and the like or a mixture thereof. The reaction can be carried out at a temperature between about −10° C. to about 45° C. The duration of reaction can be maintained for a period in the range of about 5 minutes to about 2 hours, for example about 30 minutes.

The compound of formula (Ib) was converted to a compound of formula (Ic) in presence of ammonia or aqueous ammonia solution, where all symbols are as defined earlier. The solvent used in the reaction can be selected from dichloromethane, dichloroethane, pyridine, chloroform, tetrachloromethane, ethylacetate, methanol, ethanol, isopropanol, n-propanol, butanol, acetone, acetonitrile, dimethylformamide, dimethylsulfoxide, tetrahydrofuran, water and the like or a mixture thereof. The reaction can be carried out at a temperature between about 10° C. to about 45° C. The duration of reaction can be maintained for a period in the range of about 1 hour to about 10 hours, for example about 6 hours.

The compound of formula (Ic) was reacted with the compound of formula (Id); wherein X represents a leaving group such as halogen atom, p-toluenesulfonate, methanesulfonate, trifluoromethane sulfonate or the like and R₁ is selected from alkyl having 1 to 5 carbon atoms and is for example but not limited to methyl, ethyl, n-propyl, iso-propyl or n-butyl, the other symbols are as defined earlier; to produce a compound of formula (Ie). The solvent used in the reaction can be selected from dichloromethane, dichloroethane, pyridine, chloroform, tetrachloromethane, ethylacetate, methanol, ethanol, isopropanol, n-propanol, butanol, acetone, acetonitrile, dimethylformamide, dimethylsulfoxide, tetrahydrofuran, water and the like or a mixture thereof. The reaction can be carried out at a temperature between about 10° C. to about 45° C. The duration of reaction can be maintained for a period in the range of about 1 hour to about 10 hours, for example about 6 hours.

The compound of formula (Ie) was converted to compound of formula (If), where all symbols are as defined earlier. The solvent used in the reaction can be selected from dichloromethane, dichloroethane, pyridine, chloroform, tetrachloromethane, ethylacetate, methanol, ethanol, isopropanol, n-propanol, butanol, acetone, acetonitrile, dimethylformamide, dimethylsulfoxide, tetrahydrofuran, water and the like or a mixture thereof. The hydrolysis can be carried out in presence of acid or base for example in presence of base such as sodium hydroxide, potassium hydroxide and the like. The reaction can be carried out at a temperature between about 25° C. to about 65° C. The duration of reaction can be maintained for a period in the range of about 25 minutes to about 2 hours, for example about 45 minutes.

### Process 3

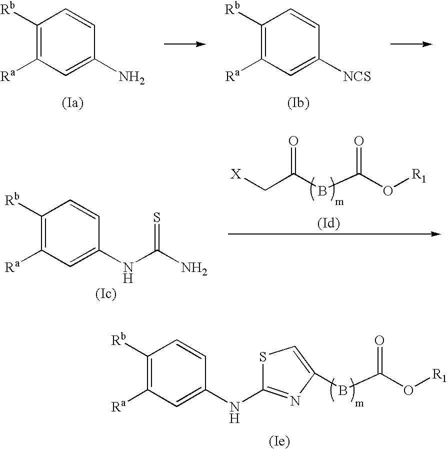

The compound of formula (Ia) was converted to a compound of formula (Ib) in presence of thiophosgene and pyridine, where all symbols are as defined earlier. The solvent used in the reaction can be selected from dichloromethane, dichloroethane, pyridine, chloroform, tetrachloromethane, ethylacetate, methanol, ethanol, isopropanol, n-propanol, butanol, acetone, acetonitrile, dimethylformamide, dimethylsulfoxide, tetrahydrofuran, water and the like or a mixture thereof. The reaction can be carried out at a temperature between about −10° C. to about 45° C. The duration of reaction can be maintained for a period in the range of about 5 minutes to about 2 hours, for example about 30 minutes.

The compound of formula (Ib) was converted to a compound of formula (Ic) in presence of ammonia or aqueous ammonia solution, where all symbols are as defined earlier. The solvent used in the reaction can be selected from dichloromethane, dichloroethane, pyridine, chloroform, tetrachloromethane, ethylacetate, methanol, ethanol, isopropanol, n-propanol, butanol, acetone, acetonitrile, dimethylformamide, dimethylsulfoxide, tetrahydrofuran, water and the like or a mixture thereof. The reaction can be carried out at a temperature between about 10° C. to about 45° C. The duration of reaction can be maintained for a period in the range of about 1 hour to about 10 hours, for example about 6 hours.

The compound of formula (Ic) was reacted with the compound of formula (Id); wherein X represents a leaving group such as halogen atom, p-toluenesulfonate, methanesulfonate, trifluoromethane sulfonate or the like and R₁ is selected from alkyl having 1 to 5 carbon atoms and may be but is not limited to methyl, ethyl, n-propyl, iso-propyl or n-butyl, the other symbols are as defined earlier; to produce a compound of formula (Ie). The solvent used in the reaction can be selected from dichloromethane, dichloroethane, pyridine, chloroform, tetrachloromethane, ethylacetate, methanol, ethanol, isopropanol, n-propanol, butanol, acetone, acetonitrile, dimethylformamide, dimethylsulfoxide, tetrahydrofuran, water and the like or a mixture thereof. The reaction can be carried out at a temperature between about 10° C. to about 45° C. The duration of reaction can be maintained for a period in the range of about 1 hour to about 10 hours, for example about 6 hours.

The following Preparation, Examples and Experimental Examples are merely illustrative, and compounds of the present application are not limited by the following embodiments in any case. A person skilled in the art can implement the present application at maximum by variously altering not only the following Examples but also claims of the present application, and such the alterations are included in claims of the present application.

### Preparation 1

### Preparation of 1-chloro-4-isothiocyanato-2-trifluoromethyl-benzene

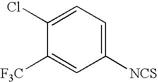

Thiophosgene (5 mL) was added portion wise to a stirred solution of 4-chloro-3-trifluoromethyl-phenylamine (10 grams) and pyridine (12.5 mL) in dichloromethane (2 Litres) and the reaction mixture was maintained at 0° C. for 30 minutes. The reaction mixture was diluted with dichloromethane and washed several times with a saturated solution of copper sulfate, followed by water and dried over sodium sulfate and evaporated to give the crude product with was further purified by passing through a column of silica gel, to yield the title compound (11 grams).

### Preparation 2

### Preparation of (4-Chloro-3-trifluoromethyl-phenyl)-thiourea

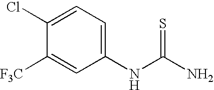

To a stirred aqueous ammonia (100 mL) was added a solution of 1-chloro-4-isothiocyanato-2-trifluoromethyl-benzene (5 grams) in acetone (10 mL) and stirred at 25-35° C. for 6 hours. The title compound obtained was filtered, dried and used for further reactions.

### EXAMPLE 1

### Preparation of [2-(4-Chloro-3-trifluoromethyl-phenylamino)-thiazol-4-yl]-acetic acid ethyl ester

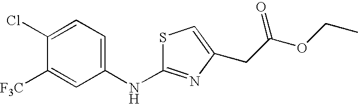

4-Chloro-3-oxo-butyric acid ethyl ester was added to a suspension of (4-chloro-3-trifluoromethyl-phenyl)-thiourea (3 grams), obtained in preparation 2, in ethanol (20 mL) and refluxed for 12 hours. The reaction mixture was cooled and the solid obtained was filtered and dried to obtain the pure title product (4.1 grams). Melting Point: 150-152° C.

MS.: 364 (M⁺) ¹H NMR (DMSO-d₆): 10.95 (br, 1H), 8.34 (d, J=2.4 Hz, 1H), 7.83 (dd, J=2.8 Hz, 8.8 Hz, 1H), 7.61 (d, J=8.8 Hz, 1H), 7.69, (s, 1H), 4.11 (q, J=7.2 Hz, 2H), 3.67 (s, 2H), 1.20 (t, J=7.2 hz, 3H).

### EXAMPLE 2

### Synthesis of [2-(4-Chloro-3-trifluoromethyl-phenylamino)-thiazol-4-yl]-acetic acid

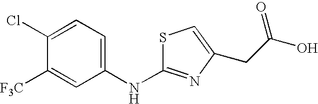

[2-(4-Chloro-3-trifluoromethyl-phenylamino)-thiazol-4-yl]-acetic acid ethyl ester (4 grams), obtained in example 1, was dissolved in minimum ethanol and 10% Sodium hydroxide (30 mL) was added and stirred at 25-35° C. for 45 minutes. The reaction mixture was then diluted with water and acidified with 10% HCl. The solid that precipitated was filtered washed with water and dried to obtain the title product. (3.2 grams). Melting Point: 158-160° C. MS.: 293 (M⁺−CO₂+1) ¹H NMR (DMSO-d₆ δ): 12.30 (br, 1H), 10.61 (br, 1H), 8.27 (d, J=2.8 Hz, 1H), 7.82 (dd, J=2.8 Hz, 9.2 Hz, 7.61 (d, J=9.2, 1H), 6.76 (s, 1H), 3.57 (s, 2H) I.R. (cm⁻¹): 3422, 1608, 1444, 1134, 1028.

### EXAMPLE 3

### Synthesis of 2-[2-(4-Chloro-3-trifluoromethyl-phenylamino)-thiazol-4-yl]-N-methyl-acetamide

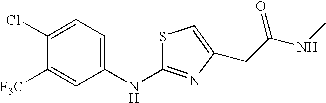

[2-(4-Chloro-3-trifluoromethyl-phenylamino)-thiazol-4-yl]-acetic acid ethyl ester (150 mg), as obtained in example 1, was dissolved in acetone and aqueous methylamine (6 mL, 40%) was added and stirred at 25-35° C. for 2 hours. Ice was added to the reaction mixture to precipitate the product (100 mg), which was washed with petroleum ether and dried. Melting Point: 148-150° C. MS.: 350 (M⁺+1) ¹H NMR (DMSO-d₆): 10.60 (s, 1H), 8.31 (d, J=2.8 hz, 1H), 7.87 (br, 1H), 7.78 (m, 1H), 7.6 (d, J=8.8 Hz, 1H), 6.70 (s, 1H), 3.43 (s, 2H), 2.62 (d, J=4.8 Hz, 3H). I.R. (cm⁻¹): 3423, 3285, 1615, 1030.

### EXAMPLE 4

### Synthesis of N-(4-Chloro-3-trifluoromethyl-phenyl)-2-[2-(4-chloro-3-trifluoromethyl-phenylamino)-thiazol-4-yl]-acetamide

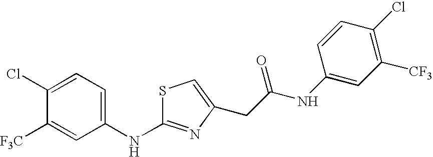

[2-(4-Chloro-3-trifluoromethyl-phenylamino)-thiazol-4-yl]-acetic acid, as obtained in example 2, was dissolved in dry dimethyl formamide, added 1-hydroxybenzotriazole (HOBT) and N-ethyl-N-(3-dimethylaminopropyl)carbodiimide (EDCI) and stirred for 30 minutes at 0° C. The corresponding aniline (1.3 eq) and N-methylmorpholine (NMM) was added and stirred at ambient temperature for 12 hours. The reaction mixture was added to ice cold water and the mater layer extracted with ethylacetate and washed with water dried over sodium sulphate and organics removed. Chromatographic purification yielded the product. Melting Point: 108-112° C. MS.: 514 (M⁺+1). ¹H NMR (CDCl₃) 9.26 (s, 1H), 7.91 (s, 1H), 7.2-7.8 (m, 5H), 6.55 (s, 1H), 3.75 (s, 2H). I.R. (cm⁻¹): 3284, 1670, 1525, 1033.

### EXAMPLE 5

### Synthesis of 2-(4-Chloro-3-trifluoromethyl-phenylamino)-thiazole-4-carboxylic acid

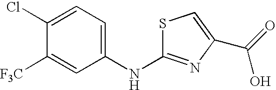

3-Bromo-2-oxo-propionic acid ethyl ester (0.59 mL) was added to a suspension of (4-chloro-3-trifluoromethyl-phenyl)-thiourea (1 gram) in ethanol (20 mL) and refluxed for 12 hours. The reaction mixture was cooled and the solid obtained was filtered and dissolved in minimum amount ethanol and 10% sodium hydroxide solution (10 mL) was added and stirred at 25-35° C. for 12 hours. The reaction mixture was then diluted with water and acidified with saturated citric acid solution. The solid that precipitated was filtered washed with water and dried to give the product (0.65 grams). Melting Point: 254-256° C. MS.: 323 (M⁺). ¹H NMR (DMSO-d₆): 12.76 (br, 1H), 10.79 (br, 1H), 8.27 (d, J=2.4 Hz, 1H), 7.94 (m, 1H), 7.82 (s, 1H), 7.66 (d, J=8.8 Hz, 1H)

I.R. (cm⁻¹): 3426, 3277, 1678, 1255.

The compounds of Example 1-a, Examples 2-a to 2-j, Examples 3-a to 3-c, and Examples 4-a to 4-n shown in the following Tables 1, 2, 3 and 4 respectively, were prepared by the same manner as described in any one of the Examples 1 to 4.

As hereunder, chemical structural formulae and physicochemical properties of Examples 1-a to 4-n are shown in Tables 1 to 4.

Symbols in the tables have the following meanings:
Ex—Example number;
DATA—physicochemical properties;
NMR—nucleomagnetic resonance spectrum;
M.S.—mass spectra;
IR—infrared spectroscopy;
Me—methyl;
Et—ethyl;
iPr—isopropyl;
Ph—phenyl.

TABLE 1
The examples given in the table below were obtained by defining various
values for R^{a}, R^{b}, R^{c}, B and m in the general formula:

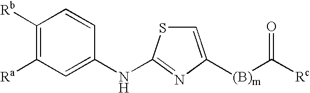

| Ex | R^{a} | R^{b} | B | m | R^{c} | Data |
|---|---|---|---|---|---|---|
| 1-a | —CF₃ | —Br | —CH2— | 1 | —O—C2H5 | Melting Point: 161-164° C. |
| | | | | | | MS.: 410 (M⁺ + 1) |
| | | | | | | ¹H NMR (DMSO-d₆): 10.86 (br, 1 H), 8.34 |
| | | | | | | (s, 1 H), 7.79 (m, 2 H), 6.79 (s, 1 H), 4.10 |
| | | | | | | (q, J = 7.2 Hz, 2 H), 3.66 (s, 2 H), 1.19 (t, |
| | | | | | | J = 7.2 Hz, 3 H). |

TABLE 2
The examples given in the table below were obtained by defining various
values for R^{a}, R^{b}, R^{c}, B and m in the general formula:

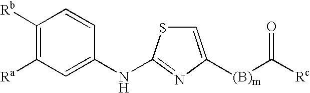

| Ex | R^{a} | R^{b} | B | m | R^{c} | Data |
|---|---|---|---|---|---|---|
| 2-a | —CF₃ | —Br | —CH2— | 1 | —OH | Melting Point: 174-178° C. |
| | | | | | | MS.: 338 (M⁺ − CO₂) |
| | | | | | | ¹H NMR (DMSO-d₆): 12.35 (br, 1 H), |
| | | | | | | 10.65 (br, 1 H), 8.28 (s, 1 H), 7.75 (m, |
| | | | | | | 2 H), 6.77 (s, 1 H), 3.58 (s, 2 H). |
| | | | | | | I.R. (cm⁻¹): 3441, 2924, 1682, 771. |
| 2-b | —Cl | —CH₃ | —CH2— | 1 | —OH | Melting Point: 142-144° C. |
| | | | | | | MS.: 283 (M⁺). |
| | | | | | | ¹H NMR (DMSO-d₆): 10.20 (br, 1 H), |
| | | | | | | 7.80 (d, J = 2.4 Hz, 1 H), 7.35 (m, 1 H), |
| | | | | | | 7.24 (d, J = 8.8 Hz, 1 H), 6.66 (s, 1 H), |
| | | | | | | 3.54 (s, 2 H), 2.25 (s, 3 H). |
| | | | | | | I.R. (cm⁻¹): 3443, 1682, 1501, 1046. |
| 2-c | —F | —F | —CH2— | 1 | —OH | Melting Point: 141-142° C. |
| | | | | | | MS.: 227 (M⁺ − CO₂). |
| | | | | | | ¹H NMR (DMSO-d₆): 12.31 9 br, 1 H 0, |
| | | | | | | 10.34 (br, 1 H 0, 7.9 (m, 1 H), 7.35 (m, |
| | | | | | | 1 H), 7.23 (m, 1 H), 6.69 (s, 1 H), 3.56 (s, |
| | | | | | | 2 H). |
| | | | | | | I.R. (cm⁻¹): 3279, 3088, 1683. |
| 2-d | —CH₃ | —CH₃ | —CH2— | 1 | —OH | Melting Point: 142-143° C. |
| | | | | | | MS.: 219 (M⁺ − CO₂). |
| | | | | | | ¹H NMR (MSO-d₆): 2.27 (br, 1 H), 9.91 |
| | | | | | | (br, 1 H), 7.34 (d, J = 2.4 Hz, 1 H), 7.25 |
| | | | | | | (m, 1 H), 7.04 (m, 1 H), 6.57 (s, 1 H), 3.52 |
| | | | | | | (s, 2 H), 2.20 (s, 3 H), 2.15 (s, 3 H). |
| | | | | | | I.R. (cm⁻¹): 3448, 2925, 1703, 1437. |
| 2-e | —F | —CH₃ | —CH2— | 1 | —OH | Melting Point: 147-148° C. |
| | | | | | | MS.: 267 (M⁺ + 1), 223 (M⁺ − CO₂). |
| | | | | | | ¹H NMR (DMSO-d₆): 12.31 (br, 1 H), |
| | | | | | | 10.22 (br, 1 H), 7.61 (dd, J = 1.6 Hz, |
| | | | | | | 12.8 Hz, 1 H), 7.13 (m, 2 H), 6.66 (s, 1 H), |
| | | | | | | 3.55 (s, 2 H), 2.16 (d, J = 1.2 Hz, 3 H). |
| | | | | | | I.R. (cm⁻¹): 3266, 3096, 1692, 1442. |
| 2-f | —OCH₃ | —OCH₃ | —CH2— | 1 | —OH | Melting Point: 135-137° C. |
| | | | | | | MS.: 250 (M⁺ + 1). |
| | | | | | | ¹H NMR (DMSO-d₆): 12.27 9 br, 1 H), |
| | | | | | | 9.90 (br, 1 H), 7.38 (d, J = 2.8 Hz, 1 H), |
| | | | | | | 7.00 (dd, J = 2.8 Hz, 8.8 Hz, 1 H), 6.88 |
| | | | | | | (d, J = 8.8 Hz, 1 H), 6.56 (s, 1 H), 3.73 (s, |
| | | | | | | 3 H), 3.71 (s, 3 H), 3.51 (s, 2 H). |
| | | | | | | I.R. (cm⁻¹): 3345, 2945, 1610, 1236. |
| 2-g | —F | —Br | —CH2— | 1 | —OH | Melting Point: 176-177° C. |
| | | | | | | MS.: 331 (M⁺), 289 (M⁺ − CO₂). |
| | | | | | | ¹H NMR (DMSO-d₆): 12.33 (br, 1 H), |
| | | | | | | 10.50 (br, 1 H), 7.87 (dd, J = 2.4 Hz, |
| | | | | | | 11.6 Hz, 1 H), 7.56 (t, J = 8.4 Hz, 1 H), |
| | | | | | | 7.21 (m, 1 H), 6.74 (s, 1 H), 3.58 (s, 2 H). |
| | | | | | | I.R. (cm⁻¹): 3420, 3263, 1681, 1569. |
| 2-h | —CF3 | —F | —CH2— | 1 | —OH | Melting Point: 167-169° C. |
| | | | | | | MS.: 321 (M⁺ + 1), 277 (M⁺ − CO₂). |
| | | | | | | ¹H NMR (DMSO-d₆): 12.33 (br, 1 H), |
| | | | | | | 10.48 (br, 1 H), 8.17 (m, 1 H), 7.84 (m, |
| | | | | | | 1 H), 7.44 (m, 1 H), 6.72 (s, 1 H), 3.56 (s, |
| | | | | | | 2 H). |
| | | | | | | I.R. (cm⁻¹): 3446, 3086, 1682, 1451. |
| 2-i | —CF3 | —CH3 | —CH2— | 1 | —OH | Melting Point: 154-155° C. |
| | | | | | | MS.: 317 (M⁺ + 1). |
| | | | | | | ¹H NMR (DMSO-d₆): 12.27 (br, 1 H), |
| | | | | | | 10.34 (br, 1 H), 8.04 (d, J = 2.0 Hz, 1 H), |
| | | | | | | 7.71 (d, J = 8.4 Hz, 1 H), 7.34 (d, J = 8.4 |
| | | | | | | Hz, 1 H), 6.69 (s, 1 H), 3.55 (s, 2 H), 2.36 |
| | | | | | | (s, 3 H). |
| | | | | | | I.R. (cm⁻¹): 3421, 1684, 1135. |
| 2-j | —CF3 | —Cl | —C(CH2)2—CH2— | 1 | —OH | Melting Point: 160-165° C. |
| | | | | | | MS.: 379 (M⁺ + 1). |
| | | | | | | ¹H NMR (DMSO-d₆): 11.87 (br, 1 H), |
| | | | | | | 10.56 (br, 1 H), 8.52 (s, 1 H), 7.71 |
| | | | | | | (m, 1 H), 7.69 (m, 1 H), 6.69 (s, 1 H), 3.55 |
| | | | | | | (s, 2 H), 2.36 (s, 3 H). |
| | | | | | | I.R. (cm⁻¹): 3421, 1684, 1135. |

TABLE 3
The examples given in the table below were obtained by defining various
values for R^{a}, R^{b}, R^{c}, B and m in the general formula:

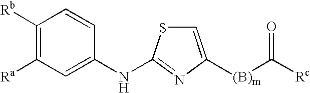

| Ex | R^{a} | R^{b} | B | m | R^{c} | Data |
|---|---|---|---|---|---|---|
| 3-a | —CF₃ | —Br | —CH2— | 1 | —NH—CH3 | Melting Point: 165-166° C. |
| | | | | | | MS.: 320 (M⁺), 322 (M⁺ + 2). |
| | | | | | | ¹H NMR (DMSO-d₆): 10.11 (br, 1 H), 8.15 (s, |
| | | | | | | 1 H), 7.2-7.8 (br m, 2 H), 7.05 (br, 1 H), 6.47 |
| | | | | | | (s, 1 H), 2.78 (d, 3 H). |
| | | | | | | I.R. (cm⁻¹): 3286, 3115, 1613, 1421, 1019. |
| 3-b | —CF₃ | —Cl | —CH2— | 1 | —NH—NH₂ | Melting Point: 115-118° C. |
| | | | | | | MS.: 350 (M⁺). |
| | | | | | | ¹H NMR (DMSO-d₆): 10.59 (br, 1 H), 9.12 |
| | | | | | | (br, 1 H), 8.21 (s, 1 H), 7.86 (d, J = 8.4 Hz, |
| | | | | | | 1 H), 7.65 (d, J = 8.4 Hz, 1 H), 6.70 (s, 1 H), |
| | | | | | | 4.24 (br, 2 H), 3.40 (s, 2 H) |
| | | | | | | I.R. (cm⁻¹): 3253, 1615, 1557. |
| 3-c | —CF₃ | —Cl | —CH2— | 1 | —NH₂ | Melting Point: 205-207° C. |
| | | | | | | MS.: 335 (M⁺ + 1). |
| | | | | | | ¹H NMR: DMSO-d₆ 10.59 (s, 1 H), 8.27 (d, |
| | | | | | | J = 2.4 Hz, 1 H), 7.84 (dd, J = 2.8 Hz, 8.8 Hz, |
| | | | | | | 1 H), 7.61 (d, J = 8.8, 1 H), 7.35 (br, 1 H), |
| | | | | | | 6.94 (br, 1 H), 6.70 (s, 1 H), 3.41 (s, 2 H) |
| | | | | | | I.R. (cm⁻¹): 3346, 2926, 1604. |

TABLE 4
The examples given in the table below were obtained by defining various
values for R^{a}, R^{b}, R^{c}, B and m in the general formula:

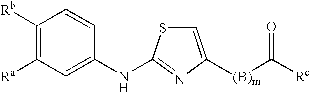

| Ex | R^{a} | R^{b} | B | m | R^{c} | Data |
|---|---|---|---|---|---|---|
| 4-a | —CF₃ | —Cl | —CH2— | 1 | 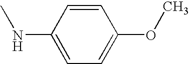 | Melting Point: 179-180° C.MS.: 442 (M⁺ + 1).¹H NMR (DMSO-d₆): 10.63 (s, 1 H),9.98 (s, 1 H), 8.25 (s, 1 H), 7.85 (d, J =6.6 Hz, 1 H), 7.4-7.6 (m, 3 H), 6.8-7.0(m, 3 H), 3.6-3.8 (m, 5 H)I.R. (cm⁻¹): 3291, 3121, 1660, 1031. |
| 4-b | —CF₃ | —Cl | —CH2— | 1 | 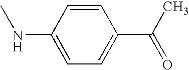 | Melting Point: 194-195° C.MS.: 454 (M⁺ + 1).¹H NMR (DMSO-d₆): 10.64 (s, 1 H),10.49 (s, 1 H), 7.24 (d, J = 2.2 Hz, 1 H),7.93 (d, J = 8.6 Hz, 2 H), 7.73-7.86 (m,3 H), 7.56 (d, J = 8.8 Hz, 2 H), 6.8 (s,1 H), 3.72 (s, 2 H).I.R. (cm⁻¹): 3337, 1662, 1599, 1406. |
| 4-c | —CF₃ | —Cl | —CH2— | 1 | 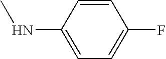 | Melting Point: 146-148° C.MS.: 430 (M⁺ + 1).¹H NMR (DMSO-d₆): 10.29 (s, 1 H),9.88 (s, 1 H), 8.15 (d, J = 2 Hz, 1 H),7.84 (m, 1 H), 7.58 (m, 2 H), 7.36 (d, J =8.8 Hz, 2 H), 6.93 (m, 2 H), 6.58 (s, 1 H),3.7 (s, 2 H).I.R. (cm⁻¹): 3297, 3125, 1666, 1031. |
| 4-d | —CF₃) | —Cl | —CH2— | 1 | 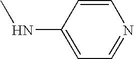 | Melting Point: 178-180° C.MS.: 413 (M⁺ + 1).¹H NMR (DMSO-d₆): 10.36 (s, 1 H),8.41 (d, J = 6.2 Hz, 2 H), 8.13 (s, 1 H),7.83 (m, 3 H), 7.61 (d, J = 6.2 Hz, 2 H),7.37 (d, J = 8.6 Hz, 1 H), 6.62 (s, 1 H),3.74 (s, 2 H)I.R. (cm⁻¹): 3441, 2926, 1690, 1196. |
| 4-e | —CF₃ | —Cl | —CH2— | 1 | 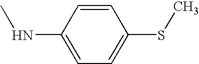 | Melting Point: 151-152° C.MS.: 458 (M⁺ + 1).¹H NMR (DMSO-d₆): 9.25 (s, 1 H), 9.09(s, 1 H), 8.23 (s, 1 H), 7.93 (s, 1 H), 7.71(d, J = 7.2 Hz, 1 H), 7.0-7.5 (m, 5 H),6.3-6.6 (m, 2 H), 3.73 (s, 2 H), 3.03 (s,3H).I.R. (cm⁻¹): 3282, 1661, 1488. 977. |
| 4-f | —CF₃ | —Cl | —CH2— | 1 | 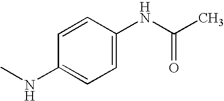 | Melting Point: 262-263° C.MS.: 469 (M⁺ + 1).¹H NMR (DMSO-d₆): 10.64 (s, 1 H),10.07 (s, 1 H), 9.86 (s, 1 H), 8.24 (s,1 H), 7.84 (d, J = 7.4 Hz, 1 H), 7.3-7.6(m, 5 H), 6.77 (s, 1 H), 3.65 (s, 2 H), 2.01(s, 3 H).I.R. (cm⁻¹): 3397, 3065, 1664, 1333. |
| 4-g | —CF₃ | —Cl | —CH2— | 1 | 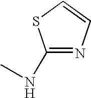 | Melting Point: 196-198° C.MS.: 419 (M⁺ + 1).¹H NMR (DMSO-d₆): 12.33 (br, 1 H),10.80 (br, 1 H), 8.24 (s, 1 H), 7.81 (s,1 H), 7.49-7.59 (m, 2 H), 7.22 (s, 1 H),6.81 (s, 1 H), 3.82 (s, 2 H).I.R. (cm⁻¹): 3438, 2925, 1573, 1324. |
| 4-h | —CF₃ | —Cl | —CH2— | 1 | 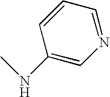 | Melting Point: 191-192° C.MS.: 413 (M⁺ + 1), . 415 (M⁺ + 3).¹H NMR (DMSO-d₆): 10.70 (s, 1 H),10.42 (s, 1 H), 8.76 (s, 1 H), 5.25-8.28(m, 2 H), 8.06 (d. J = 8.4 Hz, 1 H), 7.82(d, J = 7.4 Hz, 1 H), 7.57 (d, J = 8.6 Hz,1 H), 7.32-7.38 (m, 1H), 6.81 (s, 1 H),3.71 (s, 2 H).I.R. (cm⁻¹): 3268, 1696, 1317. |
| 4-i | —CF₃ | —Cl | —CH2— | 1 | 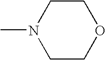 | Melting Point: 210-211° C.MS.: 406 (M⁺ + 1).¹H NMR (DMSO-d₆): 10.46 (s, 1 H), 8.22(s, 1 H), 7.82 (d, J = 8.4 Hz, 1 H), 7.62(d, J = 8.6 Hz, 1 H), 6.73 (s, 1 H), 3.71(s, 2 H), 3.35-3.54 (m, 4 H).I.R. (cm⁻¹): 3441, 3265, 1596, 1423,1030. |
| 4-j | —CF₃ | —Cl | —CH2— | 1 | 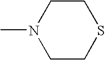 | Melting Point: 185-187° C.MS.: 422 (M⁺ + 1).¹H NMR (DMSO-d₆): 10.65 (s, 1 H),8.23 (s, 1 H), 7.84 (d, J = 8.4 hz, 1 H),7.62 (d, J = 8.8 Hz, 1 H), 6.74 (s. 1 H),3.6-3.9 (m, 5 H).I.R. (cm⁻¹): 3426, 3268, 1600, 1029. |
| 4-k | —CF₃ | —Cl | —CH2— | 1 | 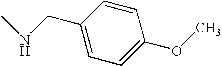 | Melting Point: 65-70° C.MS.: 456 (M⁺ + 1).¹H NMR (DMSO-d₆): 10.63 (s, 1 H),8.24 (s, 1 H), 7.86 (d, J = 8.4 Hz, 1 H),7.56 (d, J = 8.8 Hz, 1 H), 7.19 (d, J =7.6 Hz, 2 H), 7.81 (d, J = 7.6 Hz, 2 H),6.72 (s, 1 H), 4.23 (m, 2 H), 3.70 (s, 2 H),3.50 (s, 2 H).I.R. (cm⁻¹): 3423, 1649, 1517, 1130,1029. |
| 4-l | —CF₃ | —Cl | —CH2— | 1 | 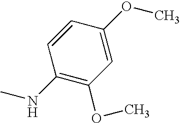 | Melting Point: 175-180° C.MS.: 456 (M⁺ + 1).¹H NMR (DMSO-d₆): 10.80 (s, 2 H),9.19 (s, 1 H), 8.33 (s, 1 H), 7.85 (m, 1 H),7.61 (d, J = 8.6 Hz, 1 H), 6.81 (s, 1 H),6.58 (s, 1 H), 6.47 (d, J = 8.6 Hz, 1 H),6.04 (m, 1 H), 3.73 (s, 1 H), 3.61 (s, 2 H).I.R. (cm⁻¹): 3412, 3067, 1529, 1042. |
| 4-m | —CF₃ | —Cl | —CH2— | 1 | 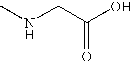 | — |
| 4-n | —CF₃ | —Cl | —CH2— | 1 | 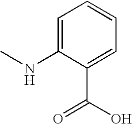 | — |

Pharmaceutically acceptable salts includes salts with inorganic bases, salts with organic bases, salts with inorganic acids, salts with organic acids, and salts with basic or acidic amino acids.

Examples of salts with inorganic bases include alkali metal salts such as sodium salt and potassium salt, alkaline earth metal salts such as calcium salt and magnesium salt, as well as aluminum salt and ammonium salt. Examples of salts with organic bases include those which are formed with trimethylamine, triethylamine, pyridine, picoline, ethanolamine, diethanolamine, triethanolamine, dicyclohexylamine and N,N′-dibenzylethylenediamine. Examples of salts with inorganic acids include those which are formed with hydrochloric acid, hydrobromic acid, nitric acid, sulfuric acid and phosphoric acid. Examples of salts with organic acids include those which are formed with formic acid, acetic acid, trifluoroacetic acid, fumaric acid, oxalic acid, tartaric acid, maleic acid, citric acid, succinic acid, malic acid, methanesulfonic acid, benzenesulfonic acid and p-toluenesulfonic acid. Examples of salts with basic amino acids include those which are formed with arginine, lysine and ornithine. Examples of salts with acidic amino acids include those which are formed with aspartic acid and glutamic acid.

A prodrug of Compound (I) refers to a compound capable of converting into Compound (I) by the action of enzymes, gastric acid and the like under in vivo physiological conditions, that is, a compound capable of converting into Compound (I) through, e.g., enzymatic oxidation, reduction and/or hydrolysis or a compound capable of converting into Compound (I) through, e.g., hydrolysis by gastric acid. Examples of a prodrug of Compound (I) include compounds obtained when an amino group of Compound (I) is acylated, alkylated or phosphorylated, such as those obtained when an amino group of Compound (I) is eicosanoylated, alanylated, pentylaminocarbonylated, (5-methyl-2-oxo-1,3-dioxolen-4-yl)methoxycarbonylated, tetrahydrofuranylated, tetrahydropyranylated, pyrrolidylmethylated, pivaloyloxymethylated or tert-butylated; compounds obtained when a hydroxy group of Compound (I) is acylated, alkylated, phosphorylated or borated, such as those obtained when a hydroxy group of Compound (I) is acetylated, palmitoylated, propanoylated, pivaloylated, succinylated, fumalylated, alanylated, dimethylaminomethyl-carbonylated or tetrahydropyranylated; and compounds obtained when a carboxyl group of Compound (I) is esterified or amidated, such as those obtained when a carboxyl group of Compound (I) is ethyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified or methylamidated. These compounds may be prepared from Compound (I) in a known manner.

The compounds of the present application are useful as activators of AMP kinase. The in vivo activation of AMPK is expected to have profound beneficial effects. It is expected that in liver, decreased expression of gluconeogenic enzymes would reduce hepatic glucose output and improve overall glucose homeostasis. Both direct inhibition and/or reduced expression of key enzymes in lipid metabolism is expected to lead to decreased fatty acid and cholesterol synthesis and increased fatty acid oxidation. Stimulation of AMPK in skeletal muscle is expected increase glucose uptake and fatty acid oxidation with resulting improvement of glucose homeostasis. It is also expected that due to a reduction in intra-myocyte triglyceride accumulation, stimulation would improved insulin action.

AMPK activation potential of the compounds of formula (I) was evaluated using a cell based ELISA approach. Skeletal muscle (L6 muscle) and hepatoma (Hep G2) liver cells were cultured for 48 hours prior to drug addition at various concentrations. Twenty four (24) hours later, the cells were fixed and the ELISA plate developed following standard protocol using p-AMPK specific antibody.

### ELISA Test:

Various cell lines such as HepG2 and L6 were revived from glycerol stocks (ATCC). The cells were maintained in a T 75 culture flask-containing medium (DMEM+10% fetal calf serum). On reaching a confluence of 70 to 80%, the cells was seeded in a 96 well plate at a density of 10×103 cells per well in DMEM+10% FCS medium. The plates are then incubated at 37° C. with 5% CO2 for 48 hours. Various concentrations of drugs were prepared in DMSO and diluted to required concentration with the medium and incubated at 37° C. with 5% CO2 for 24 hours. Cells were fixed with 4% formaldehyde in PBS for 30 minutes at 25-35° C. and washed three times with PBS containing 0.1% Triton X-100. Endogenous peroxidase was quenched with 0.6% H2O2 in PBS-T for 30 minutes and washed three times in PBS-T. Then the cells were blocked with 10% FCS in PBS-T for 1 hour. The cells were incubated for 8-12 hours with various concentrations of primary antibody in PBS-T containing 5% BSA at 4° C. The cells were then washed three times with PBS-T for 5 minutes and incubated with secondary antibody (HRP conjugated 1:500 in PBS-T with 5% BSA for 1 hour at 25-35° C. The cells were washed three times with PBS-T for 5 minutes and twice with PBS. The cells were incubated with 100 μl TMB/H2O2 color developing solution for 15 minutes and the reaction was stopped with 50 μl of 1M H2SO4. Then the plate was read at 460 nM using ELISA plate reader.

Some of the AMPK activation percentages are given in table 5 below. (AMPK activation potential percentages are obtained by normalizing the values obtained at 10 μM concentration with that of metformin by considering the values obtained for metformin at 2 mM concentrations as 100%).

TABLE 5

| | | AMPK | |
|---|---|---|---|
| | | % activation (at 10 μM) | |
| | | | HepG-2 |
| Example | | L6 muscle | muscle |
| No. | Structure | cells | cells |
| 1 | 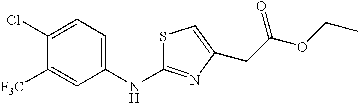 | 88.2 | 89 |
| 2 | 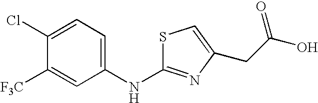 | 91.56 | 102.69 |
| 3 | 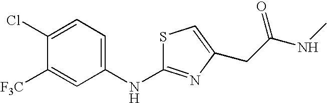 | 101.20 | 102.14 |
| 2a | 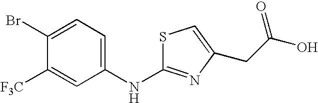 | 83.4 | 91.3 |
| 3c | 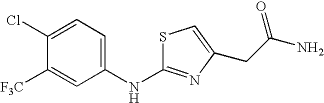 | 88.1 | 102.43 |

The pharmaceutical compositions are prepared by admixture and are suitably adapted for oral, parenteral or topical administration, and as such may be in the form of tablets, capsules, oral liquid preparations, powders, granules, lozenges, pastilles, reconstitutable powders, injectable and infusible solutions or suspensions, suppositories and transdermal devices.

Solvates of thiazole derivatives of formula (I) forming part of this application may be prepared by conventional methods such as dissolving the thiazole derivative in solvents such as water, methanol, ethanol and the like.

Hydrates of thiazole derivatives of formula (I) forming part of this application may be prepared requires the presence of water at some stage; water may be added as a co-solvent in the process. However, it is also possible to provide sufficient water for hydrate formation by carrying out the reaction with exposure to atmospheric moisture, or by use of non-anhydrous solvents.

Tablets and capsules for oral administration are usually presented in a unit dose, and contain conventional excipients such as binding agents, fillers, diluents, tableting agents, lubricants, disintegrants, colorants, flavorings, and wetting agents. The tablets may be coated according to methods known in the art.

Suitable fillers for use include cellulose, mannitol, lactose and other similar agents. Suitable disintegrants include starch, polyvinylpyrrolidone and starch derivatives such as sodium starch glycolate. Suitable lubricants include, for example, magnesium stearate. Suitable pharmaceutically acceptable wetting agents include sodium lauryl toluenesulfonate.

Solid oral compositions may be prepared by conventional methods of blending, filling, tableting or the like. Repeated blending operations may be used to distribute the active agent throughout those compositions employing large quantities of fillers. Such operations are, of course, conventional in the art.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminum stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavoring or coloring agents.

For parenteral administration, fluid unit dose forms are prepared containing a compound of the present application and a sterile vehicle. The compound, depending on the vehicle and the concentration, can be either suspended or dissolved. Parenteral solutions are normally prepared by dissolving the active compound in a vehicle and filter sterilizing before filling into a suitable vial or ampoule and sealing. Advantageously, adjuvants such as a local anesthetic, preservatives and buffering agents are also dissolved in the vehicle. To enhance the stability, the composition can be frozen after filling into the vial and the water removed under vacuum.

Parenteral suspensions are prepared in substantially the same manner except that the active compound is suspended in the vehicle instead of being dissolved and sterilized by exposure to ethylene oxide before suspending in the sterile vehicle. Advantageously, a surfactant or wetting agent is included in the composition to facilitate uniform distribution of the active compound.

What is claimed is:

1. A thiazole derivative, which is a free species and/or a pharmaceutically-acceptable salt or a solvate or a hydrate of the compound of the formula (I) 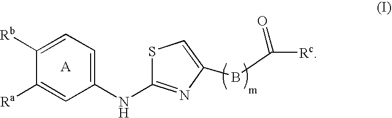
where R^{a} and R^{b}, which may be same or different, are independently chosen from fluoro, chloro, bromo, nitro, (C₁-C₅) perfluoroalkyl, (C₁-C₅) alkyl, (C₁-C₅) alkoxy, (C₁-C₅) perfluoroalkoxy, benzyl, cyano and hydroxy;
B is —CH₂, —CH(CH₃)— or —C(CH₃)₂—;
m varies between 0 and 2, inclusive;
with a proviso that R^{a} and R^{b} are not both chloro; and
R^{c} is —OR₁ or —NR₂R₃, wherein R₁ is hydrogen or (C₁-C₈) alkyl, and R₂ and R₃ are designated according to alternatives a) or b). In the alternative a), R₂ is hydrogen and R₃ is chosen from hydrogen, NH₂, (C₁-C₅) alkyl, (C₁-C₅) alkylaryl, aryl, heteroaryl, and (C₁-C₅) alkylheteroaryl. In the alternative b), R₂ and R₃, together with the nitrogen atom of the group —NR₂R₃, form a 5- or 6-membered heterocycloalkyl group.

2. The thiazole derivative of claim 1, wherein said compound has the formula (II): 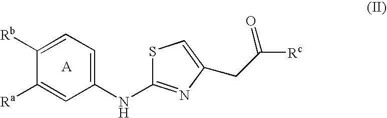
wherein R^{a}, R^{b}, and R^{c} are as defined in claim 1.

3. The thiazole derivative of claim 2, wherein R^{a} and R^{b} are independently chosen from (C₁-C₅) perfluoroalkyl, chloro, bromo, fluoro, and methyl.

4. The thiazole derivative of claim 3, wherein R^{a} is trifluoromethyl.

5. The thiazole derivative of claim 4, wherein R^{b} is chloro or bromo.

6. The thiazole derivative of claim 5, wherein said compound has the formula (III):

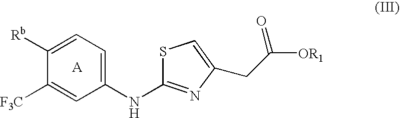

7. The thiazole derivative of claim 6, wherein R₁ is hydrogen or (C₁-C₅) alkyl.

8. The thiazole derivative of claim 6, wherein R₁ is hydrogen.

9. The thiazole derivative of claim 5, wherein said compound has the formula (IV):

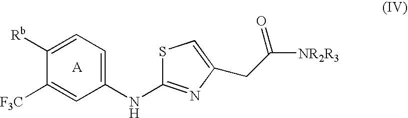

10. The thiazole derivative of claim 9, wherein R₂ is hydrogen and R₃ is (C₁-C₅) alkyl.

11. The thiazole derivative of claim 9, wherein R₂ is hydrogen and R₃ is a group of the structure 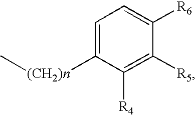
wherein R₄, R₅ and R₆ are independently chosen from chloro, bromo, fluoro, (C₁-C₅) alkyl, (C₁-C₅) alkoxy, (C₁-C₅) perfluoroalkyl, (C₁-C₅) alkylcarboxy, (C₁-C₅) alkylthio and (C₁-C₅) alkylacetomido; at least one of R₄, R₅ and R₆ is hydrogen; and n varies from 0 to 3, inclusive.

12. The thiazole derivative of claim 9, wherein R₂ and R₃, together with the nitrogen atom of the group —NR₂R₃, form a 5- or 6-membered heterocycloalkyl group.

13. The thiazole derivative of claim 12, wherein said heterocycloalkyl group includes a second heteroatom in addition to said nitrogen atom.

14. The thiazole derivative of claim 13, wherein said heterocycloalkyl group is morpholinyl or thio morpholinyl.

15. An ester prodrug of the thiazole derivative of claim 1 in accordance with the formula (I), in which R₁ is not (C₁-C₈) alkyl.

16. An ester prodrug of the thiazole derivative of claim 7 in accordance with the formula (III), in which R₁ is not (C₁-C₅) alkyl.

17. The thiazole derivative of claim 1, which has the structure

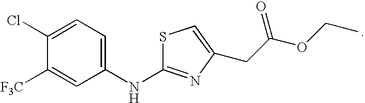

18. The thiazole derivative of claim 1, which has the structure

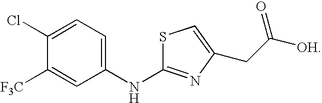

19. The thiazole derivative of claim 1, which has the structure

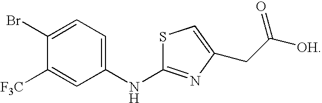

20. The thiazole derivative of claim 1, which has the structure

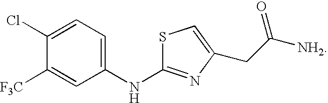

21. The thiazole derivative of claim 1, which has the structure

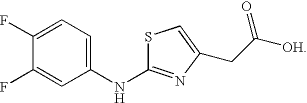

22. The thiazole derivative of claim 1, which has the structure

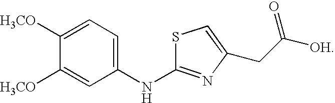

23. The thiazole derivative of claim 1, which has the structure

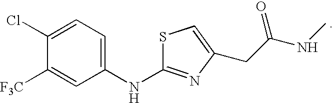

24. The thiazole derivative of claim 1, which has the structure

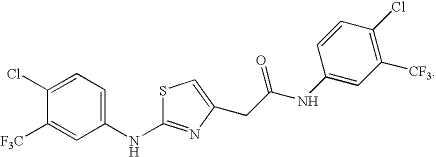

25. The thiazole derivative of claim 1, which has the structure

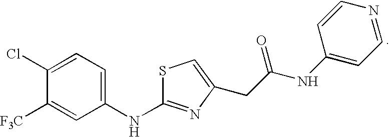

26. The thiazole derivative of claim 1, which has the structure

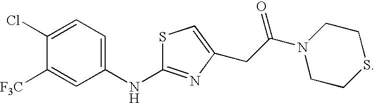

27. A thiazole derivative, which is a free species and/or a pharmaceutically-acceptable salt or a solvate or a hydrate of the compound of the formula (I) 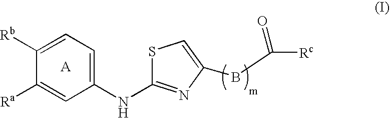
where R^{a} and R^{b} which may be same or different, are independently chosen from fluoro, chloro, bromo, nitro, (C₁-C₅) perfluoroalkyl, (C₁-C₅) alkyl, (C₁-C₅) alkoxy, (C₁-C₅) perfluoroalkoxy, benzyl, cyano and hydroxy;
B is —CH₂, —CH(CH₃)— or —C(CH₃)₂—;
m varies between 0 and 2, inclusive;
with a proviso that R^{a} and R^{b} are not both chloro; and R^{c} is —OR₁ or —NR₂R₃, wherein R₁ is hydrogen or (C₁-C₈) alkyl, and R₂ and R₃ are designated according to alternatives a) or b). In the alternative a), R₂ is hydrogen and R₃ is chosen from hydrogen, NH₂, (C₁-C₅) alkyl, (C₁-C₅) alkylaryl, aryl, heteroaryl, and (C₁-C₅) alkylheteroaryl. In the alternative b), R₂ and R₃, together with the nitrogen atom of the group —NR₂R₃, form a 5- or 6-membered heterocycloalkyl group; which thiazole derivative has AMP-activated protein kinase (AMPK) potential of at least about 80% in L6 muscle cells and of at least about 90% in Hep G2 muscle cells.

28. The thiazole derivative of claim 27, wherein R^{a} and R^{b} are independently chosen from (C₁-C₅) perfluoroalkyl, chloro, bromo, fluoro, and methyl.

29. The thiazole derivative of claim 28, wherein R^{a} is trifluoromethyl.

30. The thiazole derivative of claim 29, wherein R^{b} is chloro or bromo.

31. The thiazole derivative of claim 30, wherein said compound has the formula (III):

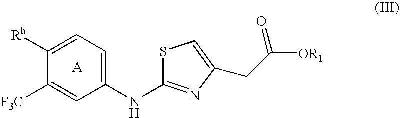

32. The thiazole derivative of claim 31, wherein R₁ is hydrogen or (C₁-C₅) alkyl.

33. The thiazole derivative of claim 31, wherein R₁ is hydrogen.

34. A method of activating AMP-activated protein kinase (AMPK) in human or animal subject, said method comprising administering said subject with an effective amount of the thiazole derivative of claim 27.

35. A pharmaceutical composition comprising one or more thiazole derivatives of claim 1 and one or more pharmaceutically-acceptable excipients.

36. A pharmaceutical composition comprising one or more thiazole derivatives of claim 27 and one or more pharmaceutically-acceptable excipients.
